SEQUENCE LISTING (20 sequences)

SEQ ID NO: 1 - protein, 18 aa, synthetic construct
GLGLTLQSLTVNDTGEYF

SEQ ID NO: 2 - protein, 18 aa, synthetic construct
SVAEHGARFQIPLLGAMA

SEQ ID NO: 3 - protein, 19 aa, synthetic construct
NADLGWHISPSFKDRVAPG

SEQ ID NO: 4 - protein, 17 aa, synthetic construct
YHTYPDGTYTGRIFLEV

SEQ ID NO: 5 - protein, 40 aa, synthetic construct
KLLSLIKGVIVHRLEGVEGSPLGLGLTLQSLTVNDTGEYF

SEQ ID NO: 6 - protein, 40 aa, synthetic construct
KLLSLIKGVIVHRLEGVEGSPLSVAEHGARFQIPLLGAMA

SEQ ID NO: 7 - protein, 41 aa, synthetic construct
KLLSLIKGVIVHRLEGVEGSPLNADLGWHISPSFKDRVAPG

SEQ ID NO: 8 - protein, 40 aa, synthetic construct
KLLSLIKGVIVHRLEGVEGSPLYHTYPDGTYTGRIFLEVE

SEQ ID NO: 9 - protein, 42 aa, synthetic construct
FNNFTVSFWLRVPKVSASHLGPSLGLGLTLQSLTVNDTGEYF

SEQ ID NO: 10 - protein, 42 aa, synthetic construct
FNNFTVSFWLRVPKVSASHLGPSLSVAEHGARFQIPLLGAMA

SEQ ID NO: 11 - protein, 43 aa, synthetic construct
FNNFTVSFWLRVPKVSASHLGPSLNADLGWHISPSFKDRVAPG

SEQ ID NO: 12 - protein, 41 aa, synthetic construct
FNNFTVSFWLRVPKVSASHLGPSLYHTYPDGTYGRIFLEVE

SEQ ID NO: 13 - protein, 18 aa, synthetic construct
KLLSLIKGVIVHRLEGVE

SEQ ID NO: 14 - protein, 20 aa, synthetic construct
FNNFTVSFWLRVPKVSASHL

SEQ ID NO: 15 - protein, 19 aa, synthetic construct
GAISLAPKAQIKESLRAEL

SEQ ID NO: 16 - protein, 41 aa, synthetic construct
KLLSLIKGVIVHRLEGVEGPSLGAISLAPKAQIKESLRAEL

SEQ ID NO: 17 - protein, 18 aa, synthetic construct
VTSEHELTCQAEGYPKAE

SEQ ID NO: 18 - protein, 40 aa, synthetic construct
KLLSLIKGVIVHRLEGVEGPSLVTSEHELTCQAEGYPKAE

SEQ ID NO: 19 - protein, 21 aa, synthetic construct
KVELMYPPPYYLGIGNGTQIY

SEQ ID NO: 20 - protein, 44 aa, synthetic construct
KLLSLIKGVIVHRLEGVEGPSLOKVELMYPPPYYLGIGNGTQIY

(19) United States
(12) Patent Application Publication
(10) Pub. No.: US 2026/0248898 A1
(43) Pub. Date: Aug. 27, 2026

(54) COMBINED TIGIT AND PD1, PDL1 AND CTLA-4 PEPTIDE VACCINES FOR IMMUNE CHECKPOINT THERAPY

(71) Applicant: The Trustees of Indiana University, Bloomington, IN (US)

(72) Inventor: Pravin T. P. Kaumaya, Fishers, IN (US)

(73) Assignee: The Trustees of Indiana University, Bloomington, IN (US)

(21) Appl. No.: 19/407,239

(22) Filed: Dec. 3, 2025

Related U.S. Application Data
(60) Provisional application No. 63/727,614, filed on Dec. 3, 2024.
(60) Provisional application No. 63/777,575, filed on Mar. 25, 2025.

(51) Int. Cl. A61K 39/00; A61P 37/04

(52) U.S. Cl. CPC A61K 39/0005; A61P 37/04; A61K 2039/585

(57) ABSTRACT

Disclosed herein is an immune checkpoint therapy utilizing the combination of a TIGIT peptide vaccine and a PDL-1 peptide vaccine, a TIGIT peptide vaccine and a PD-1 peptide vaccine, and a TGIT peptide vaccine and a CTLA-4 peptide vaccine. Also disclosed are methods of treating cancer in a subject through a dual blockade of TIGIT and PD-L1 or PD1 or CTLA-4 with combination peptide immunization of TIGIT with PD-L1, or PD1 and/or CTLA-4.

### CROSS-REFERENCE TO RELATED APPLICATIONS

The present application claims the benefit of U.S. Provisional Application No. 63/727,614, filed Dec. 3, 2024 and U.S. Provisional Application No. 63/777,575, filed Mar. 25, 2025. The contents of these applications are hereby incorporated by reference in their entirety.

### FIELD OF THE INVENTION

The present disclosure relates generally to immune checkpoint therapies for cancer treatment and the combined use of immune checkpoint inhibitors for TIGIT and PD1 or PDL1 or CTLA-4 in the immune checkpoint therapy.

### SEQUENCE LISTING

The application contains a Sequence Listing which has been submitted electronically in .xml format and is hereby incorporated by reference in its entirety. Said .xml copy, created on Dec. 2, 2025, is named IU-2025-041-03-US_st26 and is 18,879 bytes in size.

### BACKGROUND

Immune checkpoints prevent the immune response from escalating to the point that it destroys healthy cells in the body. Immune checkpoint (IC) therapy is a cancer treatment approach that blocks checkpoint proteins from binding with their partner proteins in order to prevent the “off” signal from being sent; thereby, allowing the T cells to kill cancer cells. When treated with IC inhibitors such as monoclonal antibodies (mAbs) program cell death protein/ligand 1 (PD-1/PD-L1) and cytotoxic T-lymphocyte associated protein 4 (CTLA-4), immune checkpoint therapies have improved clinical outcomes and clinical benefits for cancer patients in several cancer types. However, drug resistance and severe immune toxicity are major concerns. A small number of IC patients have developed fatal immune-related adverse events (irAEs) whereas nonfatal irAEs have improved cancer patients' outcomes.

Additionally, while checkpoint immunotherapies against cancer have gained great breakthrough in clinical benefits, the studies to date have mainly focused on the patients who well responded. More treatment options are needed to be investigated to improve personal therapeutic response rate and to avoid drugs toxicity as well as unfavorable adverse events.

In light of these issues, novel safe and non-toxic IC therapeutic strategies are needed. The present disclosure describes a novel combination of checkpoint inhibitors for use in immune checkpoint therapy that overcome the challenges described above.

### SUMMARY OF THE INVENTION

A first aspect of the invention includes a method for immune checkpoint therapy comprising a combination of a TIGIT peptide vaccine and a PD1 peptide vaccine.

A second aspect of the invention includes a method for treating a cancer in a subject comprising administration of an immune checkpoint therapy comprising a combination of a TIGIT peptide vaccine and a PDL1 peptide vaccine to the subject.

A third aspect of the invention includes a method for delaying solid tumor growth in a subject comprising administering to a subject an immune checkpoint therapy comprising combination of a TIGIT peptide vaccine and a CTLA-4 peptide vaccine.

A fourth aspect of the invention includes the immune checkpoint therapy wherein the TIGIT peptide vaccine comprises SEQ ID NO:2 or SEQ ID NO:4.

A fifth aspect of the invention includes the immune checkpoint therapy comprises a combination of TIGIT peptide vaccine, PD-L1 peptide vaccine, and CTLA-4 peptide vaccine.

A seventh aspect of the invention includes the immune checkpoint therapy wherein wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancer, small cell lung, carcinoma, non-small cell lung carcinoma, neuroblastoma, glioblastoma, ovarian cancer, pancreatic cancer, prostate cancer, skin cancer, liver cancer, melanoma, squamous cell, carcinomas of the mouth, throat, larynx, and lung, colon cancer, cervical cancer, cervical, carcinoma, breast cancer, epithelial cancer, renal cancer, genitourinary cancer, pulmonary, cancer, esophageal carcinoma, head and neck carcinoma, large bowel cancer, hematopoietic cancer; testicular cancer; prostatic cancer, or pancreatic cancer.

### BRIEF DESCRIPTION OF THE DRAWINGS

The above-mentioned and other features and advantages of this disclosure, and the manner of obtaining them, will become more apparent, and will be better understood by reference to the following description of the exemplary embodiments taken in conjunction with the accompanying drawings, wherein:

FIG. 1 is a chart of the amino acid sequences of human TIGIT and the four epitope peptides chosen for initial investigation (SEQ ID NOS: 5-12). The MVF linked peptides were used to immunize BALB/c mice, the TT3 linked peptides were used to immunize both BALB/c and C57BL/6J mice;

FIG. 2A is an image of the secondary structure of TIGIT-1(91-108) created using PyMOL 3-D modelling software Version 2.4.0 (Schrodinger, New York, NY, USA);

FIG. 2B is an image of the secondary structure of TIGIT-2(131-148) created using PyMOL 3-D modelling software Version 2.4.0 (Schrodinger, New York, NY, USA);

FIG. 2C is an image of the secondary structure of TIGIT-3(71-89)) created using PyMOL 3-D modelling software Version 2.4.0 (Schrodinger, New York, NY, USA);

FIG. 2D is an image of the secondary structure of TIGIT-4(111-129) created using PyMOL 3-D modelling software Version 2.4.0 (Schrodinger, New York, NY, USA);

FIG. 3 is the structures of TIGIT/CD155 (PMID: 22421438) and CD112 (PMID: 28515320);

FIG. 4 is a chart of the amino acid sequences of PD1, PDL1, and CTLA4 (SEQ ID NOS: 15-20, SEQ ID NO:2, SEQ ID NO:6, SEQ ID NO: 4, and SEQ ID NO: 8).

FIG. 5 is a scheme of mice vaccination and tumor engraftment. BALB/c mice (9-10 mice per group of 6-8 weeks old were immunized with MVF-peptide immunogens as indicated in the image emulsified in ISA 720 with 3 times and three weeks apart. Mice were immunized with single MVF-TIGIT 2 or 4, PDL1-Vaxx, MVF-TIGIT-2+PDL1-Vaxx or MVF-TIGIT-4+PDL1-Vaxx vaccine constructs prior to tumor challenge. Blood was collected as indicated and sera tested for antibody titers by ELISA. 2 weeks after the third immunization (3Y), the mice were engrafted with CT26 tumor cells 1×10⁵ or 4T1 tumor cells 5×10⁵ per BALB/c mouse. Control mice were treated twice weekly with PBS as negative control or with anti-mouse TIGIT antibody (clone 1G9) or anti-mouse PD-L1 antibody (clone 10F.9G2) or the anti-mouse antibody combinations as positive control starting 2 days after tumor challenge. Tumor growths were observed and measured by calipers. Tumor volume was calculated as: Tumor volume (LWW)=(Length×Width×Width)/2. The rabbits received the same vaccine immunization but without single PDL1-Vaxx and no tumor challenge. The rabbits were terminated at 3Y+3 for final sera collection for further investigations.

FIG. 6A is a graph of immunogenicity of MVF-TIGIT2/4 and PDL1-Vaxx peptides in mice or rabbits immunized with various peptide constructs.

FIG. 6B is a graph of immunogenicity of MVF-TIGIT2/4 and PDL1-Vaxx peptides in mice or rabbits immunized with various peptide constructs.

FIG. 7 is a series of plots of CT26 individual tumor volume in LWW.

FIG. 8 is a series of plots of CT26 average tumor volume in LWW.

FIG. 9 is a graph of CT26 tumor model percentage. The tumor growth inhibition (TGI %) was defined as the difference between median tumor volume (MTV) of treatment group with the PBS control group and the value was calculated by formula: TGI %=100*(MTV control-MTV test)/MTV control which were calculated at different days as indicated in the graphs.

FIG. 10 is a series of plot graphs of CT26 tumor volume in LWW at day 12, day 14, and day 16 of each group.

FIG. 11 is a series of graphs of the log-rank (Mantel-Cox) test used to compare the survival curves in multiple groups compared with PBS group. One-way ANOVA post analysis compared with PBS group, p>0.05 indicates no statistical significance, * p<0.05, ** p<0.01.

FIG. 12 is a series of plots of 4T1 individual tumor volume in LWW.

FIG. 13 is a series of plots of 4T1 average tumor volume in LWW.

FIG. 14 is a series of plots of CT26 tumor volume in LWW at day 12, day 14, and day 16 of each group.

FIG. 15 is a series of Log-rank (Mantel-Cox) test used to compare the survival curves in multiple groups compared with PBS group.

FIG. 16 is a series of Log-rank (Mantel-Cox) test used to compare the survival curves in multiple groups; compared with PBS group. For further analyze which therapy is more effective, we combined CT26 and 4T1 models by regardless of tumor model to check the overall mice survival rate.

FIG. 17 is a graph of the survival rate of mice under the same treatment to get to day 26.

FIG. 18 is a series of plots of D2F2 individual tumor volume in LWW.

FIG. 19 is a series of plots of D2F2 average tumor volume in LWW.

FIG. 20 is a series of plots of D2F2 tumor volume in LWW at day 35, day 42, and day 49 of each group.

FIG. 21 is a series of log-rank (Mantel-Cox) test plots used to compare the survival curves in multiple groups compared with PBS group * p<0.05, ** p<0.01 compared with PBS group.

FIG. 22 is a scheme of mice vaccination and tumor engraftment. BALB/c mice (9-10 mice per group) of 6-8 weeks old were immunized with MVF-peptide immunogens as indicated in the images emulsified in ISA 720 with 3 times and three weeks apart. Mice were immunized with single MVF-TIGIT 2 or 4, PD1-Vaxx or CTLA4-Vaxx, MVF-TIGIT-2+PD1-Vaxx or CTLA4-Vaxx and MVF-TIGIT-4+PD1-Vaxx or CTLA4-Vaxx, vaccine constructs prior to tumor challenge. Blood was collected as indicated and sera tested for antibody titers by ELISA. 2 weeks after the third immunization (3Y), the mice were engrafted with CT26 tumor cells 1×10⁵ or 4T1 tumor cells 5×10⁵ per BALB/c mouse. Control mice were treated twice weekly with PBS as negative control or with anti-mouse TIGIT antibody (clone 1G9) or anti-mouse PD-1 antibody (clone 29F.1A12) or anti-mouse CTLA-4 antibody (clone 9H10) or the anti-mouse antibody combinations as positive control starting 2 days after tumor challenge. Tumor growths were observed and measured by calipers. Tumor volume was calculated as: Tumor volume (LWW)=(Length×Width×Width)/2.

FIG. 23 is a graph of antigenicity of immunized mice sera against recombinant human TIGIT, PD-1 or CTLA-4 proteins via ELISA. The values of antigenicity titers are defined as the highest dilution of sera with an absorbance value of 0.2 after subtracting the blank.

FIG. 24 is a graph of Sera tittered against each individual MVF-TIGIT, PD1-Vaxx and CTLA4-Vaxx peptide immunogen by ELISA. Titers are defined as the highest dilution of sera with an absorbance value of 0.2 after subtracting the blank.

FIG. 25A-25D is a pie graph of polyclonal antibodies isotyping of immunized mice sera.

FIG. 26A is a series of plots of CT26 individual tumor volume in LWW.

FIG. 26B is a series of plots of 4T1 individual tumor volume in LWW.

FIG. 27A is a series of plots of CT26 average tumor volume in LWW.

FIG. 27B is a series of plots of 4T1 average tumor volume in LWW.

FIG. 28A is a series of plots of CT26 tumor volume in LWW at day 12 and day 14 of each group. The one-way ANOVA and post analysis was used to compared each group with PBS group.

FIG. 28B is a series of plots of CT26 tumor volume in LWW at day 12 and day 14 of each group. The one-way ANOVA and post analysis was used to compared each group with PBS group.

FIG. 29A is a series of plots of 4T1 tumor volume in LWW at day 12 and day 14 of each group. The one-way ANOVA and post analysis was used to compared each group with PBS group.

FIG. 29B is a series of plots of 4T1 tumor volume in LWW at day 12 and day 14 of each group. The one-way ANOVA and post analysis was used to compare each group with PBS group.

FIG. 30A is a series of graphs of the Log-rank (Mantel-Cox) test used to compare the survival curves in multiple groups (CT26) compared with PBS group. The ns indicates no statistical significance, * p<0.05, ** p<0.01 compared each group with PBS group.

FIG. 30B is a series of graphs of the Log-rank (Mantel-Cox) test used to compare the survival curves in multiple groups (4T1) compared with PBS group. The ns indicates no statistical significance, * p<0.05, ** p<0.01 compared each group with PBS group.

### DETAILED DESCRIPTION

The present disclosure reveals newly designed and synthesized B-cell epitope peptide vaccines targeting TIGIT (T cell immunoreceptor with immunoglobulin (Ig) and immunoreceptor tyrosine-based inhibitory motif (ITIM) domains).

The work described herein suggest that animals immunized with TIGIT peptide vaccines can elevate high titer polyclonal antibodies and be protected from faster tumor growth as well as earlier end point.

Also described herein is an immune checkpoint therapy utilizing the combination of a TIGIT peptide vaccine and one or more additional IC inhibitors, such as the program cell death protein/ligand 1 (PD-1/PD-L1) peptide vaccine. Preferably, dual blockade of TIGIT and PD-L1 with combination immunization includes a TIGIT peptide vaccine comprising TIGIT-2 or TIGIT-4 and PD-1 peptide vaccine.

The efficacy and safety of several embodiments of specific combinations of TIGIT-2/4 with PD1-Vaxx, or PDL1-Vaxx and/or cytotoxic T-lymphocyte associated protein 4 (CTLA-4) —Vaxx in several syngeneic models is described herein and shown in the figures. Preferably, combination immunotherapy of MVF-TIGIT-2/4 with PDL1-Vaxx suggests that the combo vaccination significantly delayed tumor growth and extended mice survival even better than monoclonal antibody combinations (1G9+10F.9G2). In the checkpoint immunotherapy described herein, TIGIT blockage can synergize with PD-1/PD-L1 blockade to enhance tumor rejection. The animal studies set out in the examples below illustrate the potential benefits of co-inhibition of TIGIT and PD-1/PD-L1 in enhancing anti-tumor immunity and improving treatment outcomes in several cancer types. In one preferred embodiment, the combination immunotherapy with TIGIT-2/4 plus PDL1-Vaxx indicated dual treatment could maximum therapeutic efficacy.

### TIGIT Peptide Vaccine

TIGIT is a relatively new check point molecule. TIGIT belongs to the type 1 poliovirus receptor (PVR) and is a member of the nectin family. TIGIT acts as a co-inhibitory receptor, widely expressed on CD4+ T cells, CD8+ T cells, and Tregs.

TIGIT peptide vaccines disclosed herein include four predicted epitopes of TIGIT —TIGIT-1 (SEQ ID NO:1), TIGIT-2 (SEQ ID NO:2), TIGIT-3 (SEQ ID NO:3), and TIGIT-4 (SEQ ID NO:4). Preferably, TIGIT epitopes, TIGIT-2 and TIGIT-4, are utilized as peptide vaccines in checkpoint immunotherapies, alone or in combination with other IC peptide vaccines, such as a PD-1 peptide vaccine.

The TIGIT B-cell epitope structures may be linked to a vaccine peptide specific to a human cancer type.

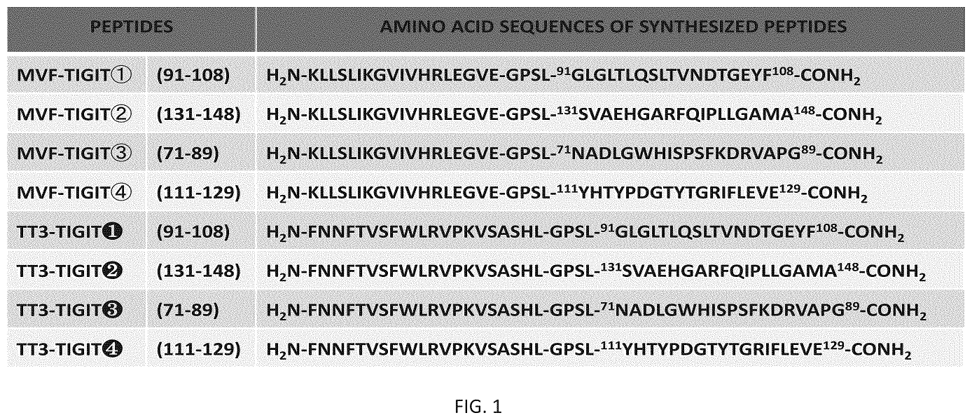

The novel B-cell epitope TIGIT peptide vaccines of the present disclosure were designed and synthesized as shown in the table of FIG. 1 (SEQ ID NO: 5 to SEQ ID NO: 12). Chimeric TIGIT B-cell peptides vaccines were made by adding a measles virus fusion peptide (MVF, amino acids 288-302, KLLSLIKGVIVHRLEGVE) (SEQ ID NO: 13) or tetanus toxoid (TT3, residue (947-967, FNNFTVSFWLRVPKVSASHL) (SEQ ID NO: 14) with a four amino acid residue (GPSL) linker to the TIGIT peptides.

Combination with Other IC Inhibitors

TIGIT peptide vaccines disclosed herein may be used on combination other IC inhibitors as a dual vaccine therapy. In some embodiments, TIGIT peptide vaccine is used in combination with a peptide vaccine for the IC inhibitor program cell death protein/ligand 1 (PD-1/PD-L1). In some embodiments, TIGIT peptide vaccine is used in combination with a cytotoxic T-lymphocyte associated protein 4 (CTLA-4) peptide vaccine. In some embodiments, TIGIT peptide vaccine is used in combination with both a PD1/PD-L1 peptide vaccine and a CTLA-4 peptide vaccine. Exemplary peptides include PD-1 92-110 (SEQ ID NO:15), PD-L1(130) (SEQ ID NO:17), and CTLA-4(130) (SEQ ID NO: 19)

### Cancer Therapy

TIGIT peptide vaccines disclosed herein may be used alone or in combination with other IC peptide vaccines, such as a PD-1 peptide vaccine, in IC cancer therapies. Applicable cancer types include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancer, small cell lung carcinoma, non-small cell lung carcinoma, neuroblastoma, glioblastoma, ovarian cancer, pancreatic cancer, prostate cancer, skin cancer, liver cancer, melanoma, squamous cell carcinomas of the mouth, throat, larynx, and lung, colon and colorectal cancers, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancer; testicular cancer; prostatic cancer, or pancreatic cancer.

Preferably, TIGIT peptide vaccines disclosed herein used, alone or in combination with other IC peptide vaccines, may be used in the treatment of solid tumor cancers including lung cancer, colon cancer, pancreatic cancer, and breast cancer.

### Formulations, Dose and Administration

The treatment of the present disclosure may comprise administering a first immune checkpoint inhibitor peptide vaccine, such as a TIGIT peptide vaccine, and a second immune checkpoint inhibitor peptide vaccine, such as a PD-L1 peptide vaccine.

The first and second vaccine compositions may be administered separately or at the same time, or in a mixture of the first and second peptide vaccine.

Each of the vaccines or the mixture of the vaccines may be administered by intravenous injection, muscle injection, subcutaneous administration, or intraperitoneal administration and can be prepared as various formulations in appropriate dosage forms depending on the administration route. The administration method and dosage form can be appropriately selected by those skilled in the art depending on the gender, age, body weight, symptoms, and the like of the patient.

For example, a patient may be vaccinated via intravenous injection three times with each dose three weeks apart from the prior dose. Each immunization may comprise a dose of the first vaccine from 0.5 mg/ml, 0.75 mg/ml, or 1 mg/ml to 1.25 mg/ml, 1.5 mg/ml, or 2 mg/ml, or any range using any two of the foregoing values as endpoints, such as 0.5 mg/ml to 2 mg/ml, or 0.75 mg/ml to 1.5 mg/ml, or 1 mg/ml to 1.25 mg/ml, and a dose of the second vaccine from 0.5 mg/ml, 0.75 mg/ml, or 1 mg/ml to 1.25 mg/ml, 1.5 mg/ml, or 2 mg/ml, or any range using any two of the foregoing values as endpoints, such as 0.5 mg/ml to 2 mg/ml, or 0.75 mg/ml to 1.5 mg/ml, or 1 mg/ml to 1.25 mg/ml.

The first and second peptide vaccine may be administered to a patient in a ratio from 1:1; 1:2; 2:1, 1:3, 3:1, 1:4, or 4:1, or any range of ratios using any two of the foregoing values as endpoints.

The vaccine composition of the present invention can be formulated according to general knowledge in the art, including pharmaceutically acceptable carriers or additives. For example, when used as an injectable formulation, the conjugate of the present invention, for example, dissolved in a solution. Alternatively, it may be freeze-dried to form a dosage form to be dissolved for reconstruction before use.

Hereinafter, the present invention will be specifically described with reference to examples.

### EXAMPLES

### Example 1: Development of TIGIT Peptide Vaccines

To provide more therapeutic options against cancer, the pre-clinical study of the present disclosure developed four human TIGIT B-cell epitope structural peptide vaccines, TIGIT-1 (91-108) SEQ ID NO:1, TIGIT-2 (131-148) SEQ ID NO:2, TIGIT-3 (71-89) SEQ ID NO:3, and TIGIT-4 (111-129) SEQ ID NO:4.

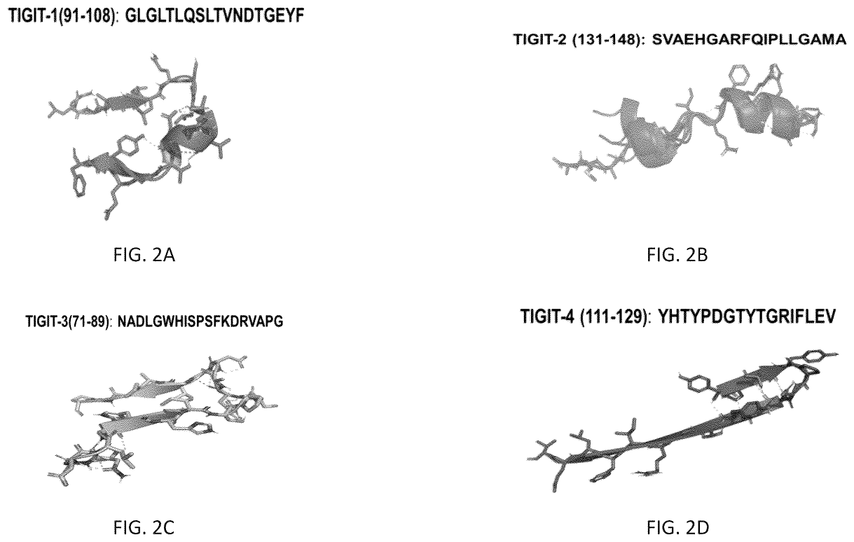

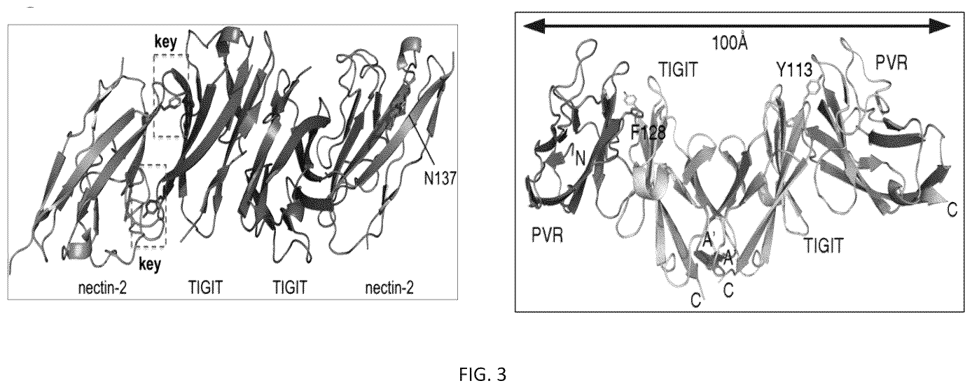

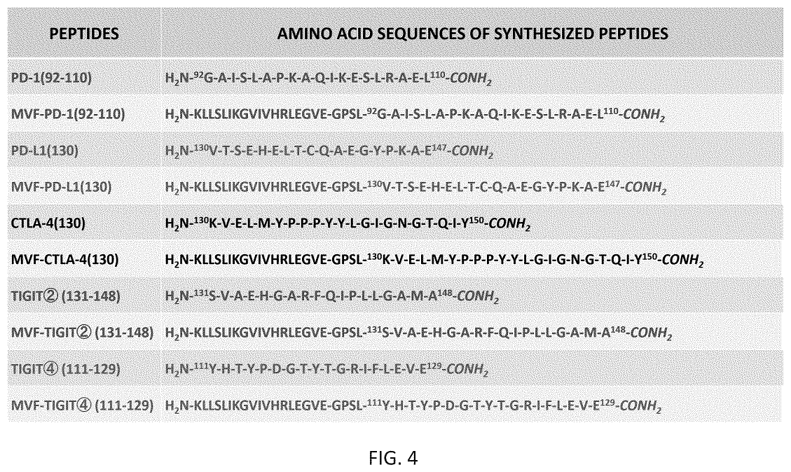

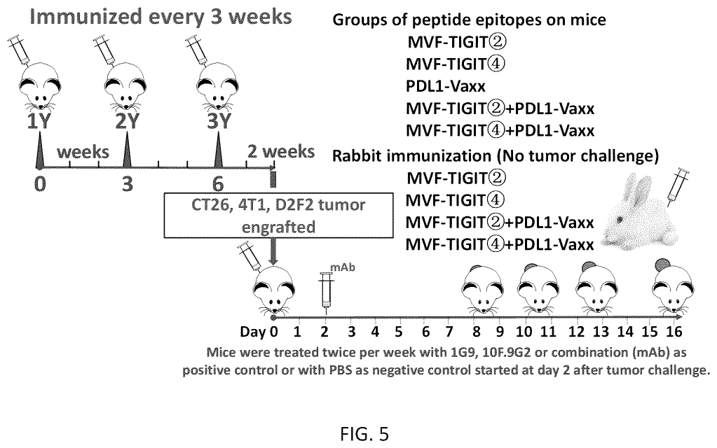

For the murine studies, the TIGIT B-cell epitope sequence is linked to a vaccine peptide specific to the mouse model used. The “promiscuous” MVF linked vaccine is designed for BALB/c and C57BL/6J mouse while tetanus toxoid (TT3) linked vaccine is mainly used on C57BL/6J mouse. Preferably, TIGIT-2 and TIGIT4 displayed overall protection of immunized mice from larger tumor burden and earlier death in multiple models (CT26, 4T1 and D2F2) especially melanoma (B16-F10). The novel B-cell epitope TIGIT peptide vaccines for the murine studies were designed and synthesized as shown in FIG. 1, FIG. 2A-2D, FIG. 3 and FIG. 4.

### Example 2: Combination Immunotherapy of TIGIT Peptide Vaccine with PDL1-Vaxx in Mouse Models

Combination immunotherapy studies were performed to verify the effectiveness of MVF-TIGIT-2 and MVF-TIGIT-4 in combination with other IC inhibitors, such as PDL1. Each rabbit received MVF-TIGIT-2, MVF-TIGIT-4, MVF-TIGIT-2+PDL1-Vaxx or MVF-TIGIT-4+PDL1-Vaxx to generate enough antibodies for further analysis. PDL1-Vaxx is shown in FIG. 4 and SEQ ID NO: 18.

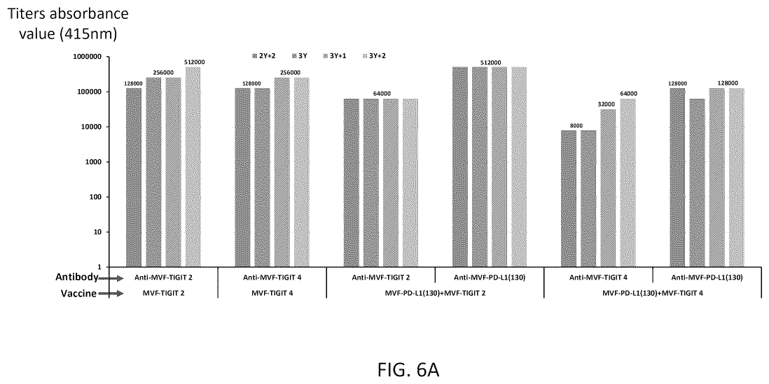

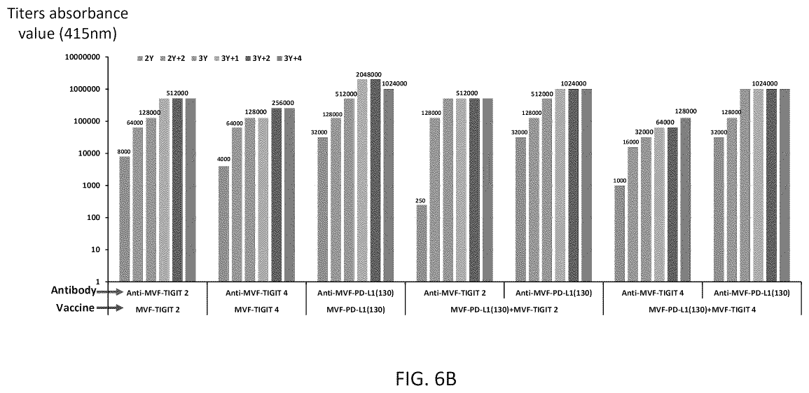

For the mouse tumor models, the CT26, 4T1 and D2F2 BALB/c syngeneic models were used. The mouse in each group was immunized with single MVF-TIGIT-2, MVF-TIGIT-4 or PDL1-Vaxx, or combination vaccines MVF-TIGIT-2+PDL1-Vaxx or MVF-TIGIT-4+PDL1-Vaxx for three times with three-week intervals. (FIG. 5). The single or combo treatment of monoclonal antibodies of anti-TIGIT and anti-PD-L1 were used as positive control and PBS as negative control. The immunogenicity of mice and rabbits were tested by ELISA that indicate the immunized animals had produce high titers of polyclonal antibodies (FIG. 6A and FIG. 6B).

The syngeneic BALB/c (CT26, 4T1 and D2F2) and C57BL/6J (MC38 and B16-F10) tumor models were used to verify anti-cancer ability by immunizing the animals with single or combination peptide vaccines. In the syngeneic colon cancer models, BALB/c CT26 and C57BL/6J MC38, the single immunization with MVF-/TT3-TIGIT peptide vaccine showed tumor growth inhibition but not in the MC38 model, while the vaccinated mice prolonged lives span especially TIGIT-2/4 that are equivalent with monoclonal antibody 1G9. To further investigate TIGIT-2/4 anti-cancer efficacy, BALB/c mammary tumor (4T1 and D2F2) and C57BL/6J melanoma (B16-F10) tumor models were used.

### Example 3: Analysis of TIGIT Peptide Vaccine in the CT26 Colon Carcinoma Tumor Model

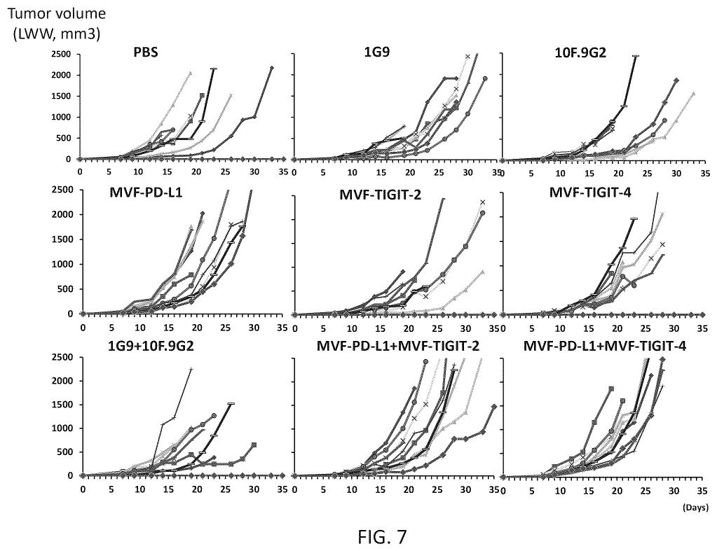

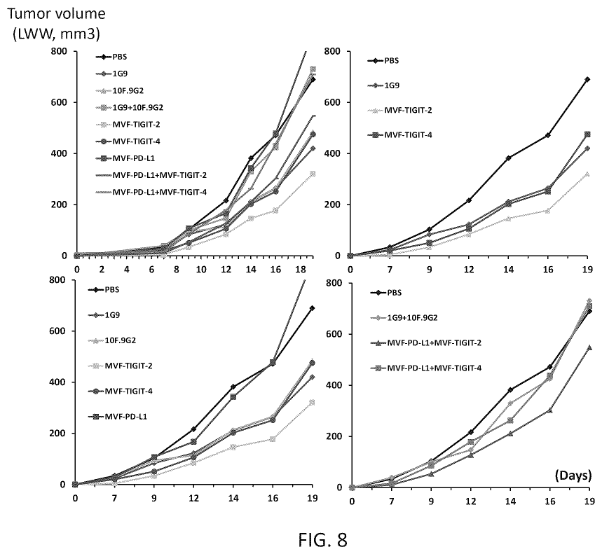

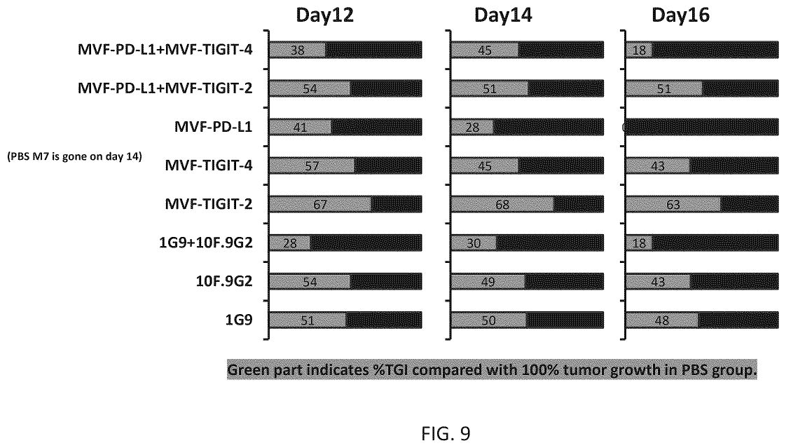

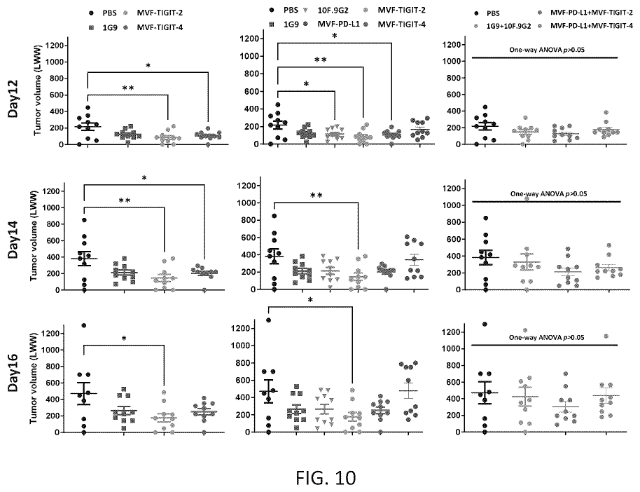

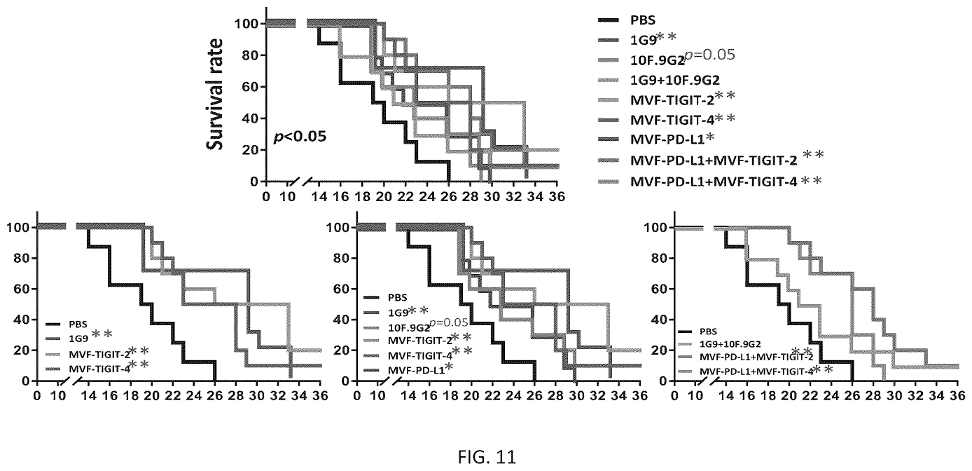

The individual tumor growth in each group (FIG. 7) and average tumor volume (FIG. 8) suggests the majority of combination immunized mice can delay tumor growth and last a longer time, but majority of PBS group mice have been sacrificed due to bad body condition or met the end points. In-depth percentage of tumor growth inhibition (% TGI) (FIG. 9) and plots (FIG. 10) on day 12, day 14 and day 16 analysis indicated single MVF-TIGIT-2 can significantly inhibit tumor growth followed by MVF-TIGIT-2+PDL1-Vaxx combination immunotherapy. The further survival rates analysis revealed that all the vaccine immunized mice showed extended survive, especially the combination immunized mice were significantly survival longer than the PBS group, but mAbs combination did not show different with PBS mice (FIG. 11).

### Example 4: Analysis TIGIT Peptide Vaccine in the 4T1 Mammary Tumor Model

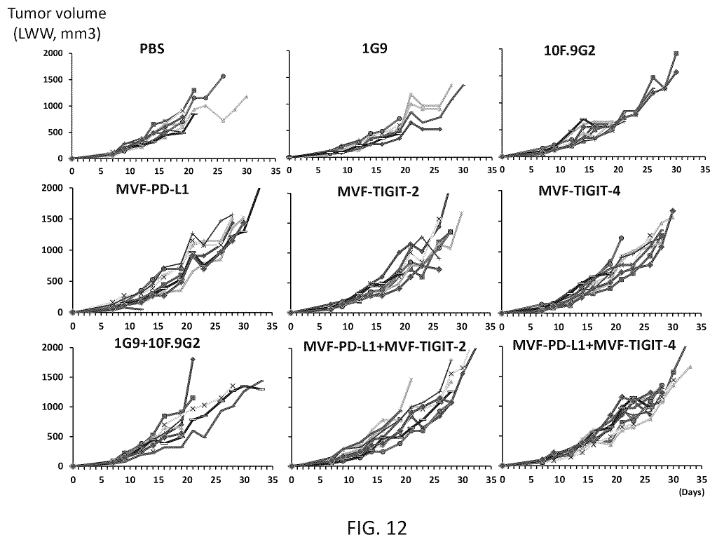

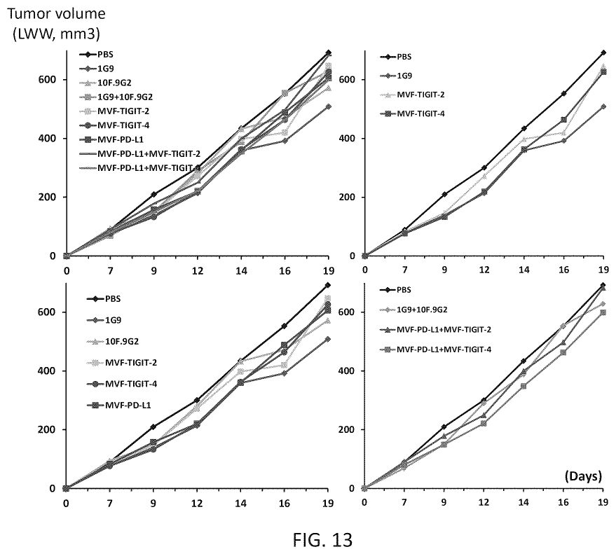

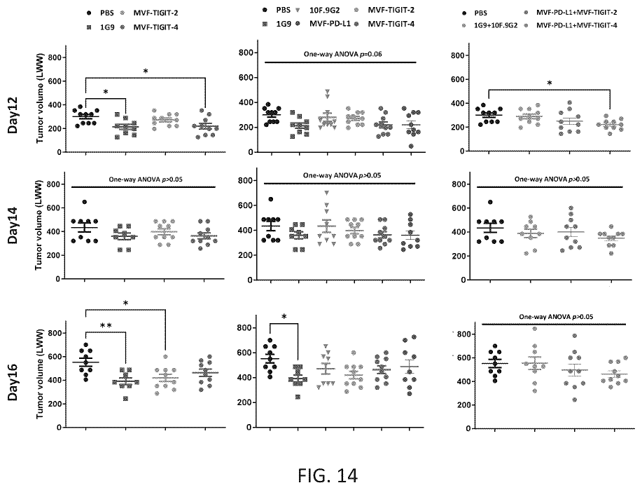

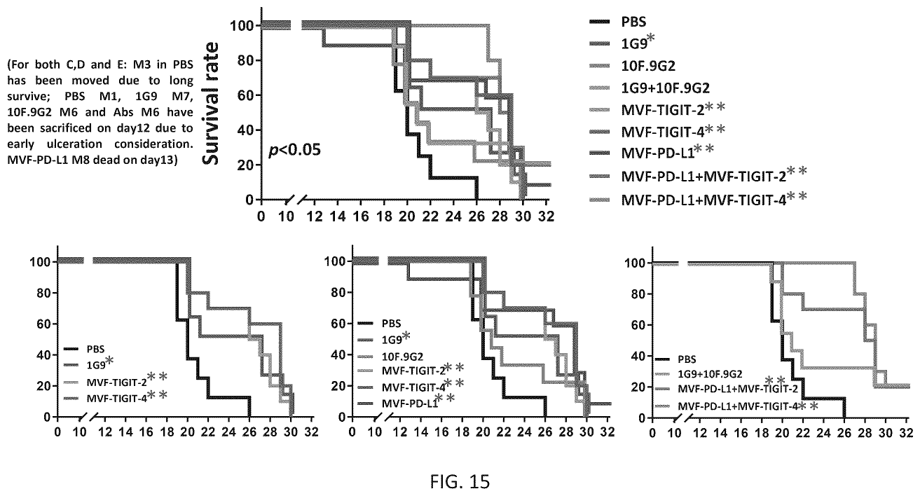

The individual 4T1 tumor growth in each group (FIG. 12) and average tumor volume (FIG. 13) indicates most combination immunized mice can delay tumor growth and last a longer time for up to over 30 days after tumor challenge, but majority of PBS group mice have been sacrificed due to bad body condition or met the end points around day 20. In-depth plots (FIG. 14) on day 12, day 14 and day 16 analysis indicated single MVF-TIGIT-4 and its combination with PDL1-Vaxx can significantly inhibit tumor growth followed by MVF-TIGIT-2+PDL1-Vaxx combination immunotherapy. The further survival rates (FIG. 15) analysis revealed that all the vaccine immunized mice showed extended survive, especially the combination immunized mice were significantly survival longer than the PBS group, but mAbs combination did not show different with PBS mice.

### Example 5: Further Analysis of TIGIT Peptide Vaccine Regardless of Tumor Models (Combine CT26 and 4T1 Models)

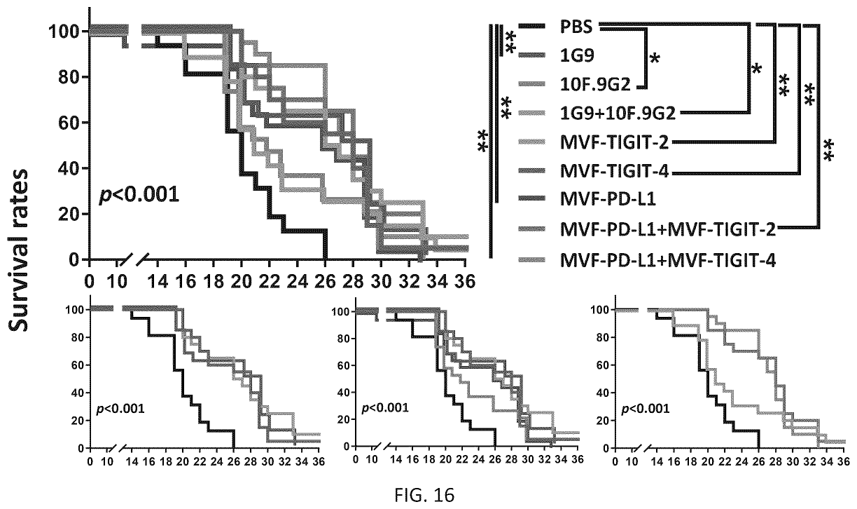

Regardless the tumor models, all the mice in the same treatment groups are combined to check the overall survival rates (FIG. 16). As we can see all the vaccine immunized mice showed extended survive, especially the combination immunized mice were significantly survival longer than the PBS group, but mAbs combination did not show different with PBS mice.

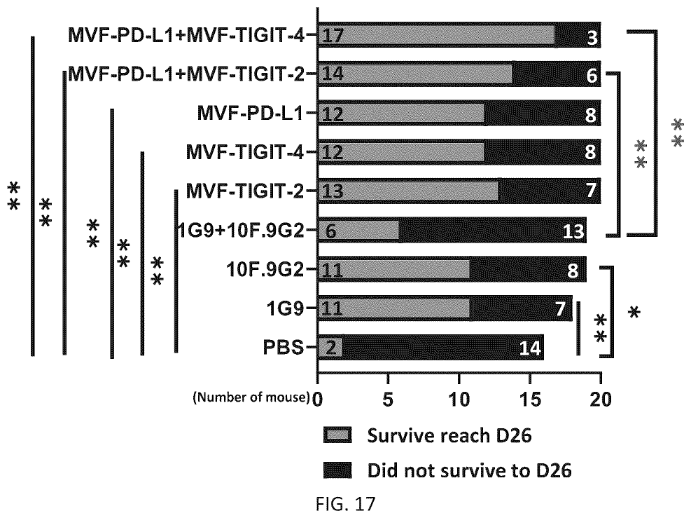

In the same group of the mice are divided as survive reach day 26 or didn't survive to day 26 after tumor challenge (FIG. 17) to perform Fisher's exact test. Compared with PBS group, the results indicate that all mice in the immunization groups and single mAb treated groups show significant difference. Furthermore, the mice in the combination vaccination groups (MVF-PD-L1 plus MVF-TIGIT-2 or MVF-TIGIT-4) have the most mice survive reach day 26 after tumor challenge that are statistically significant different from mAbs combination group.

Overall, MVF-PD-L1 plus MVF-TIGIT-2 or MVF-TIGIT-4 combined immunization are the best approaches to keep mice survive to day 26 after tumor challenge.

### Example 6: Analysis of TIGIT Peptide Vaccine in D2F2 Breast Carcinoma Model

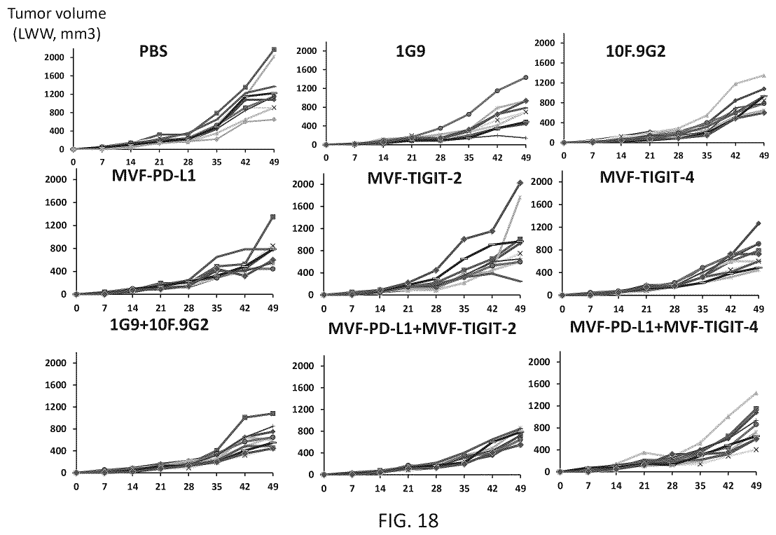

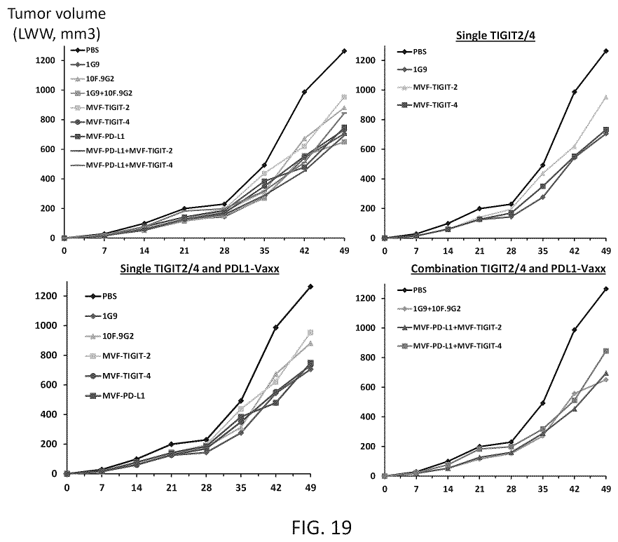

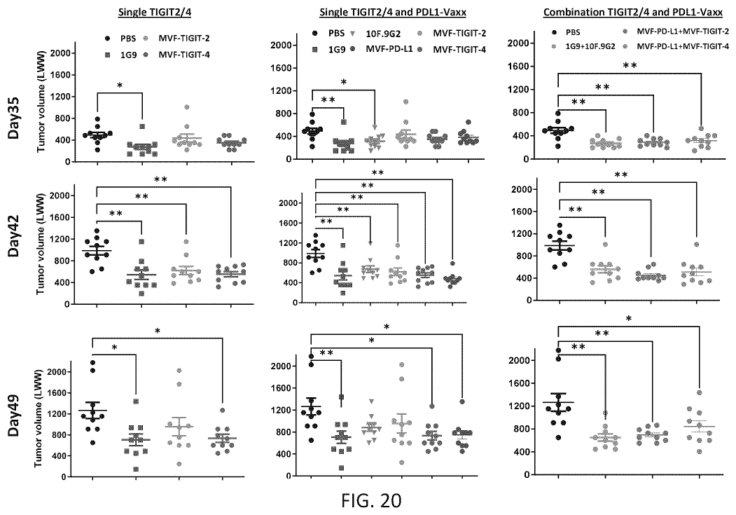

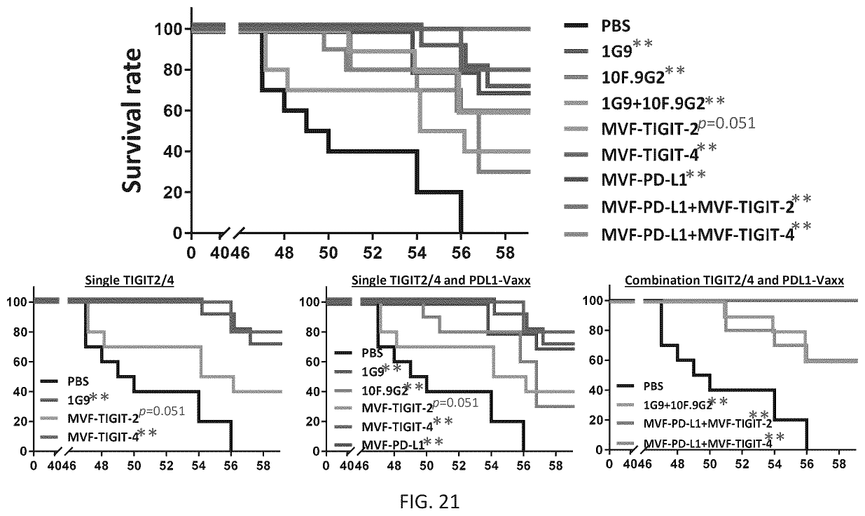

The individual D2F2 breast cancer growth in each group (FIG. 18) and average tumor volume (FIG. 19) indicate the vaccine immunization as well as the monoclonal antibody treatment can delay tumor growth and last a longer time for up to 49 days after tumor challenge, but majority of PBS group mice have been euthanized due to bad body condition or met the end points. In-depth analysis plots (FIG. 20) on day 35, day 42 and day 49 indicated single MVF-TIGIT-2 and MVF-TIGIT-4 and especially their combination with PDL1-Vaxx can significantly inhibit tumor growth. The further survival rates (FIG. 21) analysis revealed that the vaccine immunized mice showed dramatically extended survive, especially the MVF-TIGIT-2 with PDL1-Vaxx combination immunized mice none of the mouse meet the end point.

### Example 7: Further TIGIT Peptide Vaccine Combination Immunotherapy Studies

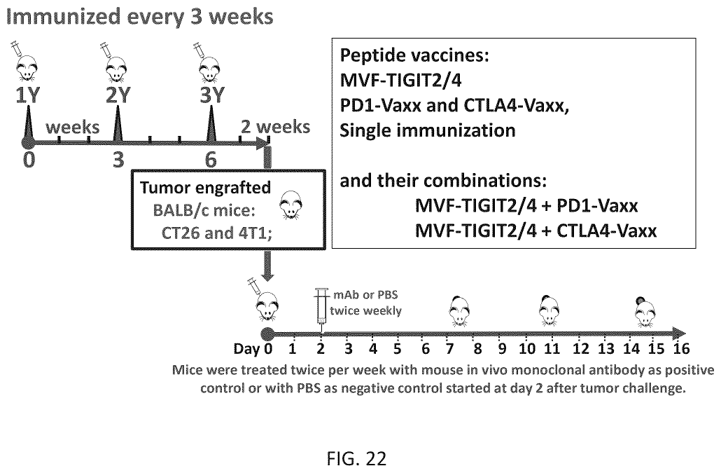

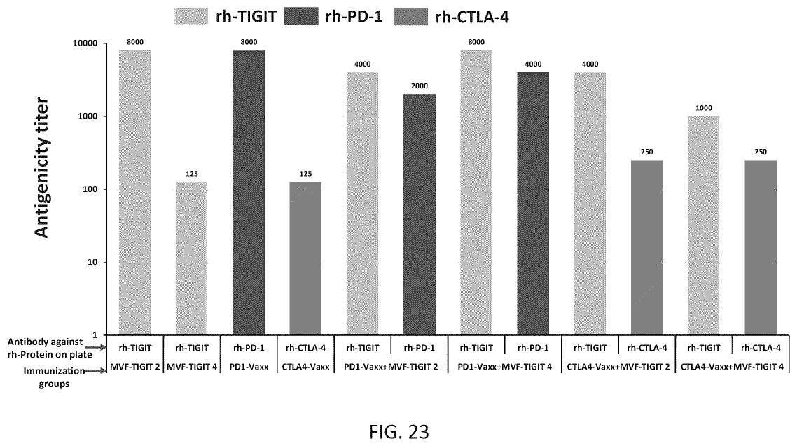

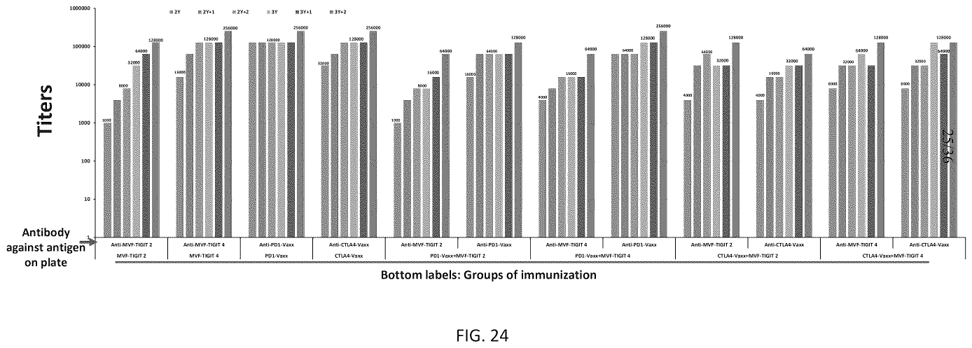

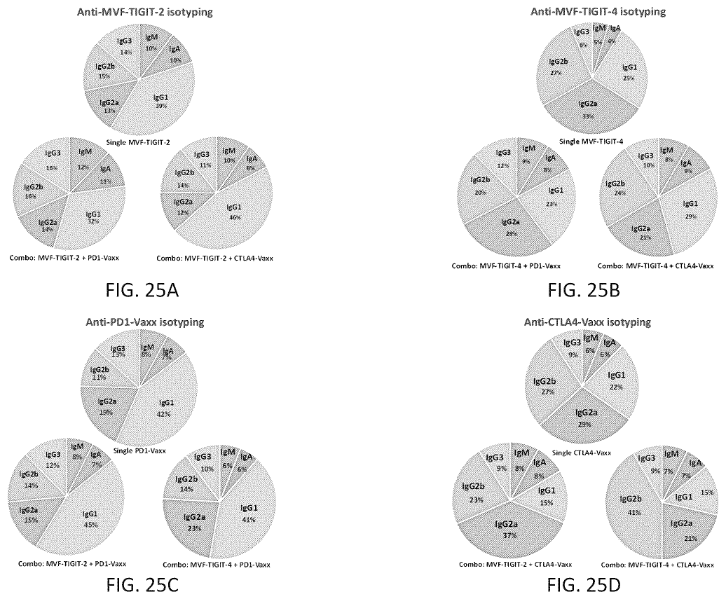

Further combination immunotherapy studies were performed to verify the effectiveness of MVF-TIGIT-2 and MVF-TIGIT-4 in the combination with PD1-Vaxx or CTLA4-Vaxx in syngeneic mouse tumor models. The BALB/c mouse CT26 and 4T1 tumor models were used. The mouse in each group was immunized with single MVF-TIGIT-2, MVF-TIGIT-4, PDL1-Vaxx or CTLA4-Vaxx, or combination vaccines MVF-TIGIT-2 or MVF-TIGIT-4+PDL1-Vaxx or CTLA4-Vaxx for three times with three-week intervals. The sequence for CTLA4-Vaxx is shown in FIG. 4 and SEQ ID NO:20. The single or combo treatment of monoclonal antibodies of anti-TIGIT, anti-PD-L1 and anti-CTLA-4 were used as positive control and PBS as negative control (FIG. 22). The antigenicity against recombinant human TIGIT, PD-1 or CTLA-4 proteins (FIG. 23) and immunogenicity activities to its immunogens (FIG. 24) of immunized mice sera suggested the vaccination boost high titers of polyclonal antibodies showed the across binding ability to recombinant human proteins. As shown in FIGS. 25A-25D, the major subtypes of anti-MVF-TIGIT2 polyclonal antibodies are IgG1, followed by IgG2a/2b and IgG3. While the most subtype of anti-MVF-TIGIT4 and anti-PD1-Vaxx are still IgG1 with less IgG2a and IgG2b but did not have much IgG3 subtype compared with anti-MVF-TIGIT2. For the polyclonal antibodies isotyping of anti-CTLA4-Vaxx, IgG2a ranked top in MVF-TIGIT-2 plus CTLA4-Vaxx combination with IgG2b was the top in MVF-TIGIT-4 plus CTLA4-Vaxx combination. IgG2a and IgG2b are about the same (29% of IgG2a and 27% of IgG2b) in single CTLA4-Vaxx followed with 22% of IgG1.

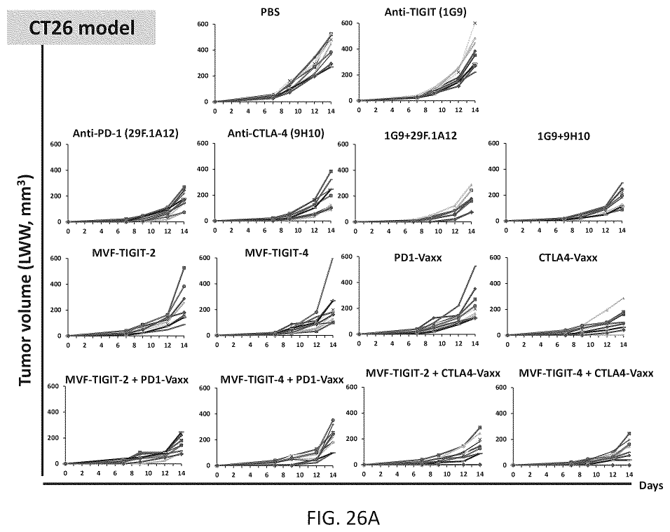

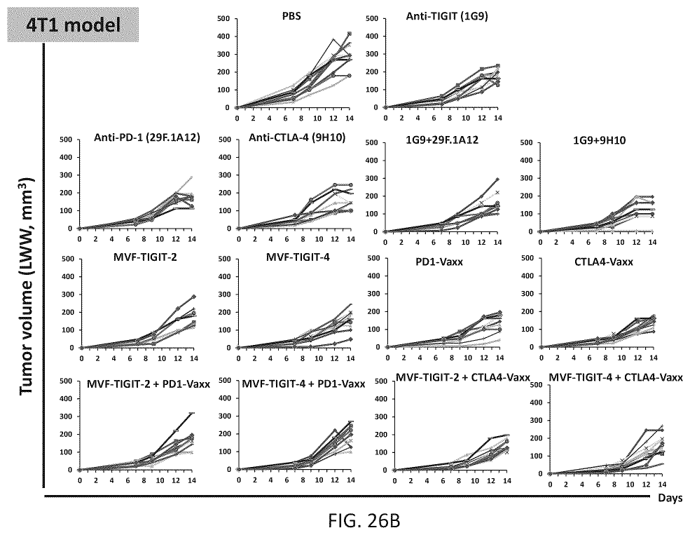

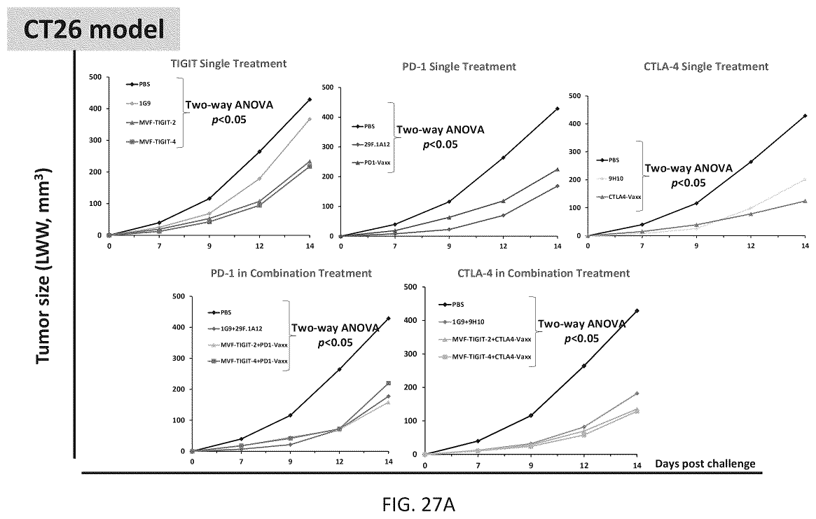

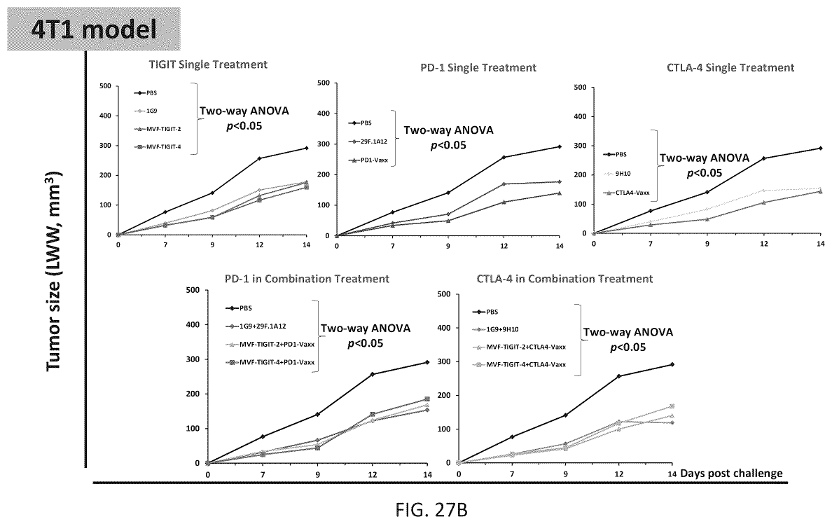

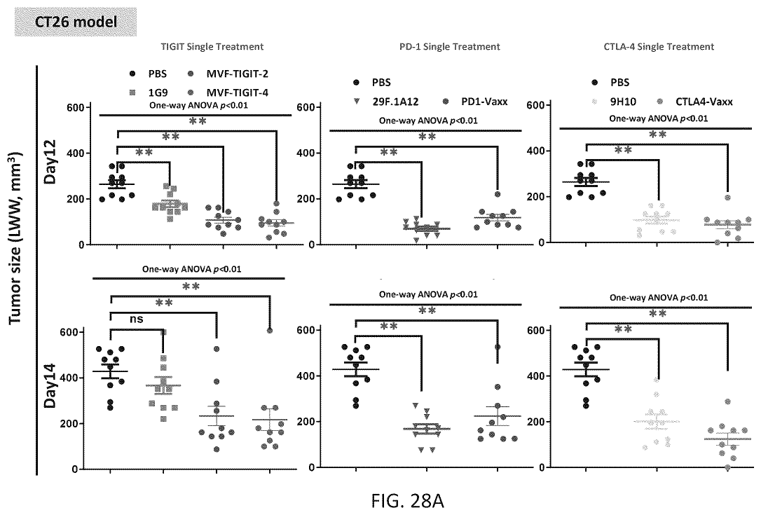

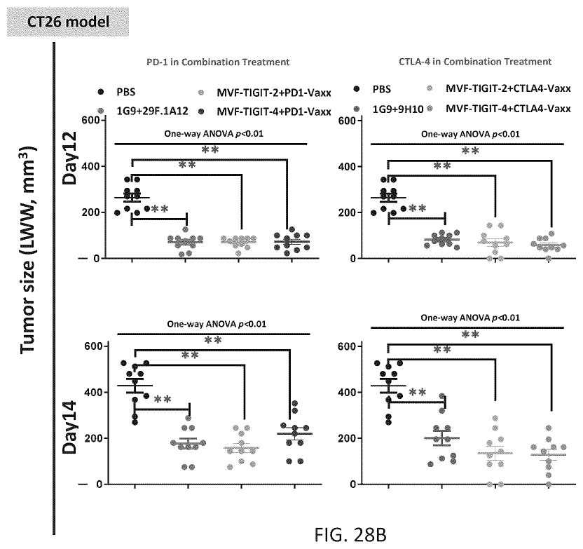

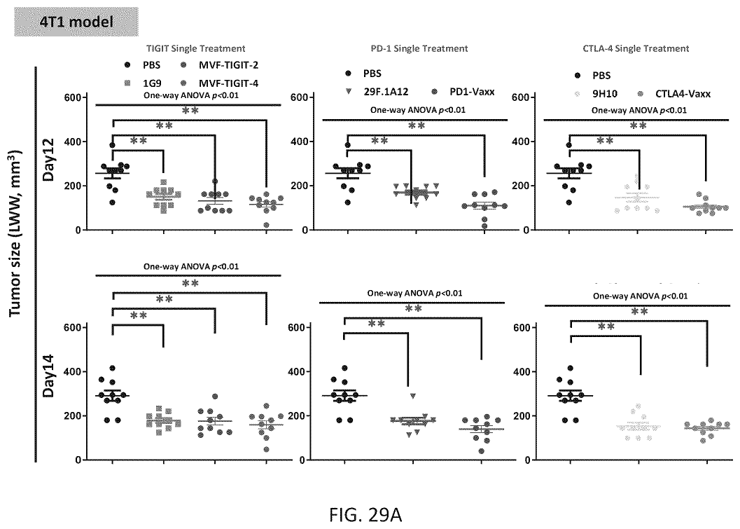

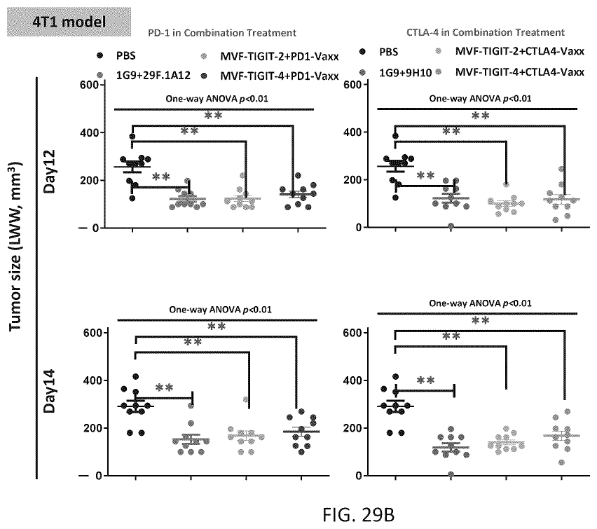

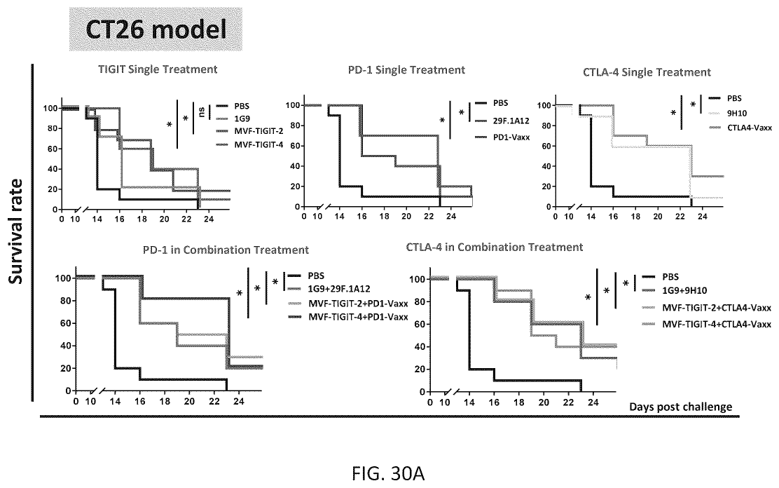

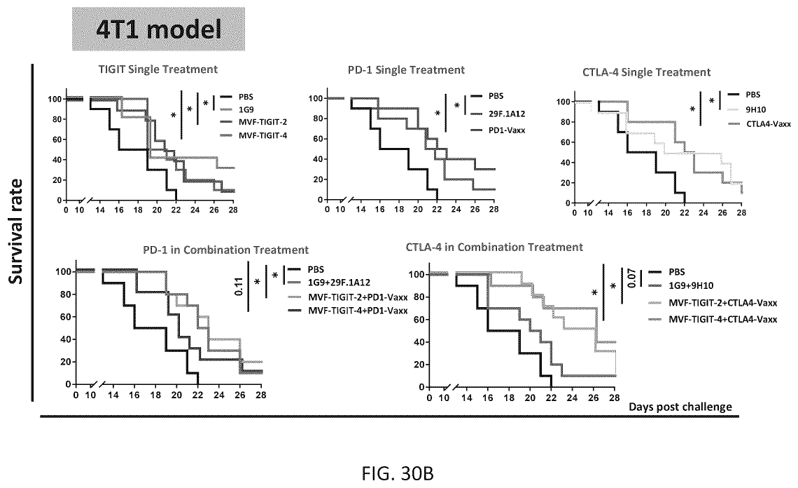

The individual CT 26 and 4T1 tumor growth in each group (FIGS. 26A and 26B) and average tumor volume (FIGS. 27A and 27B) indicate single or combination immunized as well as most of monoclonal antibodies treated mice can delay tumor growth challenge. In-depth (FIGS. 28A, 28B, 29A, and 29B) on day 12 and day 14 analysis indicated except single anti-TIGIT mAb in CT26 model, all the monoclonal treated groups as well as single or combination vaccination groups can significantly inhibit tumor growth. Accordingly, all the groups except anti-TIGIT mAb treatment in the CT26 tumor model extended mice survival rates compared with PBS control. Vaccination and mAb treatment prolonged mouse survival rates with 4T1 tumor as well, instead of two groups that MVF-TIGIT-4 plus PD1-Vaxx or anti-CTLA-4 mAb vs PBS show p=0.11 or p=0.07 (FIGS. 30A and 30B), respectively.

### Materials and Methods

### Peptide Synthesis

Chimeric TIGIT B-cell peptides vaccines were made by adding a measles virus fusion peptide (MVF, amino acids 288-302, KLLSLIKGVIVIRLEGVE) or tetanus toxoid (TT3, residue (947-967, FNNFTVSFWLRVPKVSASHL) with a four amino acid residue (GPSL) linker to the TIGIT peptides. Each peptide was synthesized on a Liberty Prime System (CEM Matthews, NC) using there one pot method. 500 mg of Rink Amide Protide Resin LL (CEM) was used to synthesize 0.1 mmol of peptide. Fmoc amino acids (AAPPtec Louisville, KY) were dissolved in N—N-Dimethylformamide (DMF) (Fisher Sci) at a concentration of 0.5M. Deprotection was done using a 25% v/v Pyrrolidine (Sigma-Aldrich) solution in DMF. Amino acids were activated using a combination of 0.26 M Oxyma (CEM) in DMF and 0.75 M N,N′-Diisopropylcarbodiimide (DIC) (Fisher Sci) in DMF. Deprotections were done at 110° C. and activation and synthesis were done at 105° C. Peptides were single coupled for the first 24 amino acids. All arginines were also double coupled, as well as all couplings after the 24th.

After synthesis, resins were dried and cleaved using a Razor Cleavage System (CEM). The recommended Razor Cleavage solution was used (92.5% Trifluoroacetic acid (TFA) (Fisher Sci)/2.5% w Triisopropylsilane (TIPS) (VWR Radnor, PA)/2.5% DDI water/2.5% 3,6-dioxa-1,8-octanedithiol (DODT) (Sigma-Aldrich)). Cleavages were carried out for 70 minutes at 41° C. Peptides were precipitated and washed using ice cold Diethyl Ether (Fisher Sci). Cleaved peptides were analyzed using a Waters ACR HPLC system (Waters Corporation Framingham, MA) using a BEH C18 analytical Column (Waters). Peptides were purified using a CEM Prodigy System (CEM) on a BEH C18 semi-prep Column (Waters).

### High Performance Liquid Chromatography (HPLC) Analysis

All analytical HPLC samples were Run on a Waters ARC HPLC system using a Waters BEH C18 4.6 mm×150 mm column (Waters Corp., Bedford, MA). Peptides were dissolved in 10% acetic acid in water at 2 mg/ml. Analysis were done using a gradient of ddi water 0.1% Trifluoracetic acid (TFA) (Sigma-Aldrich St. Louis, MO) using a gradient of 10-90% Acetonitrile (Fisher Scientific, Rockford, IL) 0.1% TFA at 60° C.

All peptides were purified using a Prodigy Semi-prep HPLC System (CEM). Peptides were dissolved in 10% acetic acid in water at 5 mg/ml. Purifications were done using a gradient of ddi water 0.1% TFA using a gradient of 10-90% Acetonitrile 0.1% TFA at 600C. Samples containing peptide were pooled and lyophilized using a FreeZone 4.5L Bench top Freeze dryer (Labconco, Kansas City, MO). Peptides were submitted to Indiana Biosciences Research Institute (Indianapolis, IN) for LC-MS analysis.

### Animals: Rabbits, C57BL/6J Mice and BALB/c Mice

All experiments were performed following the U.S. Public Health Service Policy on Humane Care and Use of Laboratory Animals experiments approved by the Ohio State University Institutional Animals Care and Use Committee as well as the Indiana University School of Medicine Laboratory Animal Resource Center (LARC). Female New Zealand white rabbits, C57BL/6J, and BALB/c mice were purchased from Charles River Laboratories (Wilmington, MA, USA). All animal care and use were in accordance with ULAR (University Laboratory Animal Resources) and LARC institutional guidelines.

### Animal Immunization

For each peptide, vaccine antibodies were raised using female New Zealand white rabbits (>2 Kg/8-10 weeks of age). All immunizations were carried out by emulsifying the synthetic peptide immunogens with Montanide ISA720 (SEPPIC Paris, France). Rabbits were immunized with 1 mg chimeric MVF or TT3 linked TIGIT peptides intramuscularly 4 points injection and boosted twice at three weeks and at six weeks. The rabbits were immunized with single or combination vaccines to test immunogenicity and generate enough sera to in-depth analysis (FIGS. 1-11).

C57BL/6J mice were immunized with 100 μg TT3 linked peptide while BALB/c mice were immunized with 100 μg MVF or TT3 linked peptides subcutaneously. The four chimeric peptide-based candidate vaccines were used to immunize all animals. BALB/c mice or C57BL/6J mice (5-6 weeks old) were immunized with chimeric peptide immunogens 3 times at 3 weeks intervals referred to as primary immunization (1Y), first boost (2Y) and second boost (3Y). The mice sera were collected every week after secondary and tertiary immunization (2Y, 2Y+1, 2Y+2, 3Y, 3Y+1 and 3Y+2). Mouse tumor models were challenged as indicated with tumor cells at 3Y+2, control mice were treated twice weekly with PBS (100 μl/mouse) as negative control or with anti-mouse TIGIT antibody (clone 1G9, 100 μl/mouse of 2 mg/ml) as positive control starting 2 days after tumor challenge, mice were treated twice per week during the experimental period. Tumor growths were observed and measured by calipers.

### Mice In Vivo Tumor Challenging Experiment

The mice were engrafted with tumor cells as indicated in each experiment scheme (detailed in each of the figure legend) after the last immunization at 3Y, then the control mice were treated with PBS or mAb two days after tumor challenging followed by tumor volume and mice survival rates monitoring.

BALB/c tumor models and all mice were challenged two weeks after the last immunization (3Y) at 3Y+2: CT26 (1×105 cells/mouse), 4T1 (5×10⁵ cells/mouse), and D2F2 (2×10⁵ cells/mouse); C57BL/6J tumor models: MC38 (1×105 cells/mouse), and B16-F10 (1×105 cells/mouse).

### Antibody Purification

The peptide vaccine antibodies from immunized rabbits were purified by affinity chromatography using a protein A/G column (Pierce brand source of ThermoFisher, Rockford, IL) and eluted antibodies (Abs) were concentrated and exchanged in Phosphate-buffered saline (PBS) using 50 kDa cutoff centrifuge filter units (MilliporeSigma, Burlington, MA, USA). The concentration was measured by spectrophotomic absorption at 280 nm.

### Cell Lines

CT26, D2F2, 4T1, B16-F10, and MC38 tumor cell lines were used in this study. CT26, MC38, B16-F10 and 4T1 cell lines were maintained in DMEM or RPMI-1640 basic medium. D2F2 cell line was maintained in DMEM with 10% NCTC-109 medium (Invitrogen, Waltham, MA, USA) and 1×MEM Non-Essential Amino Acids Solution (ThermoFisher, Rockford, IL, USA). All cell culture media were supplemented with 10% fetal bovine serum (FBS), 100 units/ml penicillin and 100 μg/ml streptomycin.

### Enzyme-Linked Immunosorbent Assay (ELISA)

Immunogenicity was evaluated by ELISA standard laboratory protocols as described in Guo L, Overholser J, Darby H, Ede N J, Kaumaya PTP: A newly discovered PD-L1 B-cell epitope peptide vaccine (PDL1-Vaxx) exhibits potent immune responses and effective anti-tumor immunity in multiple syngeneic mice models and (synergizes) in combination with a dual HER-2 B-cell vaccine (B-Vaxx). Oncoimmunology 2022, 11(1):2127691. Briefly, 96-well plates were coated with 100 μl of peptide as antigen at 2 μg/ml in ddH2O overnight at 4° C. Nonspecific binding sites were blocked for 1 h with 200 μl PBS (Research Products International, Mt Prospect, IL, USA, CAS No. 7647-145) 1% BSA (Bovine serum albumin, Thermo Fisher Scientific, Waltham, WA, USA, BP9703-100), and plates were washed with washing buffer (PBS diluted 0.05% Tween 1% horse serum). Vaccine antibodies in blocking buffer (PBS 1% BSA) were added to antigen-coated plate in duplicate wells, serially diluted 1:2 in blocking buffer, and incubated for 2 h at room temperature. After washing the plate, the secondary antibody 100 μl of 1:500 goat anti-mouse or anti-rabbit IgG conjugated to horseradish peroxidase (Invitrogen, Waltham, MA, USA, REF:31430) was added to each well and incubated for 1h. After washing, the antibody was detected using substrate solution (50 μl of 0.15% H2O2 in 24 mM citric acid and 5 mM sodium phosphate buffer (pH 5.2) with 0.5 mg/ml 2,2′-aminobis (3-ethylbenzthiazole-6-sulfonic acid, ABTS, Sigma, St. Louis, MO, USA) as the chromophore. Color development proceeded for 10 min, and the reaction was stopped with 25 μl of 1% SDS (sodium dodecyl sulfate, Thermo Scientific, Waltham, WA, USA, Prod #28312). Absorbance was read at 415 nm using an ELISA Microplate reader (Molecular Devices, SPECTRAmax PLUS384, San Jose, CA, USA).

### Recombinant Protein Activity Assay

For the detection of antibody reactivity with human recombinant TIGIT protein 1 μg recombinant protein in 100 μl of PBS or the concentration as indicated in the figures was used to coat wells overnight at 4° C. After the overnight incubation, nonspecific binding sites were blocked for 1 h with 200 μl PBS 1% BSA, and plates were washed with washing buffer (PBS diluted 0.05% Tween 1% horse serum). Vaccine antibodies in blocking buffer were added to antigen-coated plate in duplicate wells, serially diluted 1:2 in blocking buffer, and incubated for 2 h at room temperature. After washing the plate, 100 μl of 1:500 goat anti-mouse or anti-rabbit IgG conjugated to horseradish peroxidase (Invitrogen, Waltham, MA, USA, REF:31430) was added to each well and incubated for 1h. After washing, the plate received a final wash and 50 μl prepared ABTS substrate solution was added to each well (BIO-RAD, Hercules, CA, USA, Cat. #1721064). Color development proceeded for 10 min, and the reaction was stopped with 25 μl of 1% SDS. Absorbance was read at 415 nm using an ELISA Microplate reader (Molecular Devices, SPECTRAmax PLUS384, San Jose, CA, USA).

### Antibody Isotyping Assay

The assay was carried out by following the manufacturer's instructions (BIO-RAD, Mouse Typer isotyping kit, Cat. #172-2055) and lab protocol. Briefly, mouse antibody isotypes (i.e. IgA, IgM, IgG1, IgG2a, IgG2b, and IgG3) were determined using the Mouse Typer isotyping Kit (BIO-RAD, Hercules, CA, USA, Cat. #172-2055). Wells of a 96-well assay plate (COSTAR, Washington, D.C., USA, REF #2797) were coated with 100 μl of 2 μg/ml peptide antigen in ddH2O and incubated at 4° C. overnight. The plate was washed with washing buffer (0.05% tween-20 and 1% horse sera in PBS). The plate was blocked with 1% BSA in PBS at room temperature for 1h. 100 μl of diluted sera was added to each well. Dilutions of each sera samples were determined by the ELISA titers absorbance of 0.4 or higher after subtracting the background. After washing the wells, 100 μl ready to use rabbit anti-mouse subclasses antibody was added to each well respectively and incubated at room temperature for 2 h. The wells were washed again, 100 μl (1/3000 dilution of goat anti-rabbit conjugated to HRP antibody (BIO-RAD, Hercules, CA, USA, Cat. #172-1019)) was added to each well and incubated for 1 h at room temperature in dark. The plate received a final wash and 50 μl prepared ABTS substrate solution was added to each well (BIO-RAD, Hercules, CA, USA, Cat. #1721064). The reaction was stopped with 25 μl 5% SDS stopping buffer. Absorbance at 415 nm was determined using a plate reader.

### Statistical Analysis

Mice challenged with tumor cells were monitored at least twice per week and calipers were used to measuring tumor volumes. Formula: Volume (LWW)=(Length×Width×Width)/2 was used to calculate tumor volumes. Percentage of tumor growth inhibition (TGI %) was defined as the difference between median tumor volume (MTV) of treatment group with the PBS control group and the value was calculated by formula: TGI %=100*(MTV control-MTV test)/MTV control All values are showed as means±standard deviation (SD). Data statistical analysis was performed by GraphPad Prism 8.1.2 (GraphPad Software, Inc. San Diego, CA, USA) and the indicated statistical analysis. One-way analysis of variance (one-way ANOVA) and followed by the Tukey's multiple comparisons test were used to compare data in multiple groups or data between groups in multiple groups. The two-way ANOVA followed by Dunnett's multiple comparisons test was used to analysis the whole curves comparison. The Log-rank (Mantel-Cox) test was used to compare the survival curves. Fisher's exact test was used as indicated in the figure legend. P value or adjusted p value less than 0.05 was accepted as statistically significant different

SEQUENCES

| SEQ ID NO: | Name | Sequence |
|---|---|---|
| 1 | TIGIT-1 (91-108) | GLGLTLQSLTVNDTGEYF |
| 2 | TIGIT-2 (131-148) | SVAEHGARFQIPLLGAMA |
| 3 | TIGIT-3 (71-89) | NADLGWHISPSFKDRVAPG |
| 4 | TIGIT-4 (111-129) | YHTYPDGTYTGRIFLEV |
| 5 | MVF-TIGIT 1 | KLLSLIKGVIVHRLEGVE-GSPL- |
| | | GLGLTLQSLTVNDTGEYF |
| 6 | MVF-TIGIT 2 | KLLSLIKGVIVHRLEGVE-GSPL- |
| | | SVAEHGARFQIPLLGAMA |
| 7 | MVF-TIGIT 3 | KLLSLIKGVIVHRLEGVE-GSPL- |
| | | NADLGWHISPSFKDRVAPG |
| 8 | MVF-TIGIT 4 | KLLSLIKGVIVHRLEGVE-GSPL- |
| | | YHTYPDGTYTGRIFLEVE |
| 9 | TT3-TIGIT 1 | FNNFTVSFWLRVPKVSASHL-GPSL- |
| | | GLGLTLQSLTVNDTGEYF |
| 10 | TT3-TIGIT 2 | FNNFTVSFWLRVPKVSASHL-GPSL- |
| | | SVAEHGARFQIPLLGAMA |
| 11 | TT3-TIGIT 3 | FNNFTVSFWLRVPKVSASHL-GPSL- |
| | | NADLGWHISPSFKDRVAPG |
| 12 | TT3-TIGIT 4 | FNNFTVSFWLRVPKVSASHL-GPSL- |
| | | YHTYPDGTYGRIFLEVE |
| 13 | MVF, amino acids | KLLSLIKGVIVHRLEGVE |
| | 288-302 | |
| 14 | TT3, amino acids | FNNFTVSFWLRVPKVSASHL |
| | 947-967 | |
| 15 | PD-1 (92-110) | GAISLAPKAQIKESLRAEL |
| 16 | MVF-PD-1 (92-110) | KLLSLIKGVIVHRLEGVE-GPSL- |
| | | GAISLAPKAQIKESLRAEL |
| 17 | PD-L1 (130) | VTSEHELTCQAEGYPKAE |
| 18 | MVF-PD-L1 (130) | KLLSLIKGVIVHRLEGVE-GPSL- |
| | | VTSEHELTCQAEGYPKAE |
| 19 | CTLA-4 (130) | KVELMYPPPYYLGIGNGTQIY |
| 20 | MVF-CTLA-4 (130) | KLLSLIKGVIVHRLEGVE- |
| | | GPSLOKVELMYPPPYYLGIGNGTQIY |

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practices in the art to which this disclosure pertains.

I claim:

1. An immune checkpoint therapy comprising a combination of a TIGIT peptide vaccine and one or more second immune checkpoint inhibitor peptide vaccines.

2. The immune checkpoint therapy of claim 1 wherein the TIGIT peptide vaccine comprises a TIGIT epitope sequence selected from SEQ ID NO:2 or SEQ ID NO:4.

3. The immune checkpoint therapy of claim 2 wherein the one or more second immune checkpoint inhibitor vaccine is selected from group consisting of PD-1 peptide vaccine, PD-L1 peptide vaccine and CTLA-4 peptide vaccine.

4. A method of treating a cancer in a subject comprising administering to a subject an immune checkpoint therapy comprising combination of a TIGIT peptide vaccine and a PD-L1 peptide vaccine.

5. The method of claim 4 wherein the TIGIT peptide vaccine comprises SEQ ID NO:2.

6. The method of claim 4 wherein the TIGIT peptide vaccine comprises SEQ ID NO:4.

7. The method of claim 5 wherein the PD-L1 peptide vaccine comprises SEQ ID NO:17.

8. The method of claim 6 wherein the PD-L1 peptide vaccine comprises SEQ ID NO: 17.

9. The method of claim 4 wherein the immune checkpoint therapy further comprises a CTLA-4 peptide vaccine.

10. The method of claim 4 wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancer, small cell lung carcinoma, non-small cell lung carcinoma, neuroblastoma, glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancer; testicular cancer; prostatic cancer, or pancreatic cancer.

11. The method of claim 10 wherein the cancer is colon cancer.

12. The method of claim 10 wherein the cancer is breast cancer.

13. A method of delaying solid tumor growth in a subject comprising administering to a subject an immune checkpoint therapy comprising combination of a TIGIT peptide vaccine and a PD-L1 peptide vaccine.

14. The method of claim 13 wherein the TIGIT peptide vaccine comprises SEQ ID NO:2 or SEQ ID NO:4.

15. The method of claim 14 wherein the PD-L1 peptide vaccine comprises SEQ ID NO: 17.

16. The method of claim 13 wherein the solid tumor comprises colon cancer cells.

17. The method of claim 13 wherein the solid tumor comprises breast cancer cells.

18. The method of claim 13 further comprising administering a CTLA-4 peptide vaccine.
